(19) United States
(12) Patent Application Publication
(10) Pub. No.: US 2023/0329977 A1
(43) Pub. Date: Oct. 19, 2023

(54) REMINERALIZATION COMPOSITION AND APPLICATION METHOD THEREOF

(71) Applicant: ZEST IP HOLDINGS, LLC, Carlsbad, CA (US)

(72) Inventors: Yuyan JIAO, Irvine, CA (US); Wen GU, Riverside, CA (US); Yuping LI, Irvine, CA (US); Nicholas ARALIS, San Juan Capistrano, CA (US)

(21) Appl. No.: 18/134,493

(22) Filed: Apr. 13, 2023

Related U.S. Application Data
(60) Provisional application No. 63/331,506, filed on Apr. 15, 2022.

(51) Int. Cl. A61K 6/20; A61K 6/70; A61K 6/60

(52) U.S. Cl. CPC A61K 6/20; A61K 6/70; A61K 6/60

(57) ABSTRACT

Dental remineralization compositions and methods are provided herein. In an aspect of the disclosure, a remineralization composition comprises a remineralizing agent and a polyanion serving as a multi-point anchor to localize the remineralizing agent on tooth surfaces. The remineralization composition can comprise a non-ionic surfactant that promotes formation of complexes between the remineralizing agent and the polyanion. In some aspects, the polyanion comprises poly(acrylic acid) sodium salt. In some aspects, the remineralizing agent comprises at least one of bioactive calcium containing compounds, bioactive calcium containing complexes, and free calcium ions formed once the bioactive calcium containing compounds contacts or mixes with water. In some aspects, the remineralization composition is free of phosphates and/or phosphate salts.

### CROSS-REFERENCE TO RELATED APPLICATION

This application claims priority to U.S. Provisional Application Ser. No. 63/331,506, filed on Apr. 15, 2022. This and all other extrinsic materials discussed herein, including publications, patent applications, and patents, are incorporated by reference in their entirety. Where a definition or use of a term in an incorporated reference is inconsistent or contrary to the definition of that term provided herein, the definition of that term provided herein applies and the definition of the term in the reference does not apply.

### FIELD OF THE INVENTION

The field of the invention is remineralization compositions.

### BACKGROUND

Enamel is an outer layer of a tooth that protects the inner structure, including the sensitive part of the tooth. When enamel is worn, damaged or missing, teeth are more susceptible to cavities and decay, which can lead to infections, pain, sensitivity, and other problems. Everyday items such as food and drinks contain acid and are the most common cause of tooth enamel loss. Further, issues such as dry mouth, acid reflux, and low-salivary flow can contribute to tooth enamel loss. Lost enamel cannot be restored, and there is a need to prevent or reduce enamel loss and to strengthen weakened enamel.

### SUMMARY

The present disclosure relates to a remineralization composition comprising a polyanion serving as a multi-point anchor to adhere or localize remineralizing agents such as calcium ions or complexes to tooth surfaces, and application methods thereof. In some aspects, the remineralization composition is formulated for both enamel and dentin remineralization, wherein the compositions comprise bioactive compounds such as calcium compounds and/or calcium complexes, polyanions, and optionally a non-ionic surfactant. The bioactive compounds and/or complexes can serve as remineralizing agents. In some embodiments, the remineralization composition can be free of, or essentially free of, phosphates. In some embodiments, the remineralizing agent(s) of a composition can be free of, or essentially free of, phosphates. In some embodiments, the remineralizing agent(s) present in a formulation do not include amorphous calcium phosphate (ACP) or amorphous calcium fluoride phosphate (ACFP). It is contemplated that the disclosed phosphate free remineralization compositions are more effective in tooth remineralization than that of phosphate containing compositions. Both calcium and phosphate ions are required to promote remineralization. Traditionally remineralization agents are calcium-phosphate systems which contain both calcium and phosphate. The calcium-phosphate system relies on dissolution its remineralizing agents in aqueous solution or saliva in order to release calcium and phosphate ions, to which they interact, leading to deposition of a layer of hydroxyapatite on the enamel, i.e., dental calculus. Prior to hydroxyapatite layer formation on enamel surface, calcium and phosphate ions must be available temporarily for being able to diffuse into enamel to induce remineralization. It is believed the calcium-phosphate system in most cases encourages dental calculus deposition rather than remineralization. Further, it is known that calcium phosphate has low water solubility. Where phosphate salts are incorporated into a remineralization composition, the phosphate salts can react with the remineralizing agent, i.e. calcium containing salts, forming calcium phosphate precipitates/crystalized hydroxyapatite. When such compositions are applied to tooth surface, the precipitated calcium phosphate need to be re-dissolved before remineralizing enamel/dentin and therefore may form dental calculus. In some preferred compositions of the disclosure, the compositions are free or essentially free of phosphates and/or phosphate salts and thus can avoid the formation of surface calcium phosphate precipitate layer on enamel prior to remineralization treatment. Polyanions can chelate remineralizing agents and adhere to tooth surface. The compositions can slowly release of reminieralizing agent(s) at the teeth and/or penetrate the remineralizing agent(s) into enamel/dentin, resulting an effective remineralization.

Enamel, which protects the inner layers of teeth, can be strengthened and repaired through remineralization. It can occur when minerals from saliva and certain foods deposit inside enamel or dentin. Some efforts have been made to increase levels of calcium and phosphate in the immediate vicinity of the teeth to enhance the remineralization process.

Unfortunately, known remineralization products such as calcium-phosphate systems often show results no better than using 1000 ppm OTC F dentifrice. Further, it has been found that there is weak and insufficient evidence to support recommending known remineralization products. Therefore, there is a still-unmet need for compositions and methods for the effective remineralization of tooth structures including enamel and dentin.

Without wishing to be bound by any particular theory, it is contemplated that in commercial products aimed at tooth remineralization in the aqueous environment, cationic ions are released from calcium-containing complexes or rapidly exchange with hydrogen cations (in the form of H₃O⁺), which brings about the release of calcium and phosphate (PO₄³⁻) ions, thus forming a calcium phosphate layer. As these reactions continue, this layer crystallizes into carbonate-enriched hydroxyapatite (HA). This crystallized HA layer restricts the passage of ions into the deeper, more affected layers. Remineralization of dentin thus remains ineffective or unsuccessful. In an aspect of the disclosure, compositions and methods are provided for remineralization of the enamel and deeper, more affected layers of the tooth. In an aspect, the present disclosure provides a remineralization composition capable of continuous and effective remineralization not only on enamel but also in dentin. In an aspect, the remineralization composition uses a polyanion which serves as a multi-point anchor to both bind the remineralizing agents and localize the remineralizing agents on tooth surface, allowing long-term deposition of the remineralizing agent into tooth structure (underneath the outer surface of the enamel) while preventing it from surface precipitation or from being quickly washed away by saliva. In another aspect, the present disclosure provides a remineralization composition capable of slowly releasing remineralizing agents through the use of a bioadhesive polyanionic matrix complexed with cations and/or cationic complexes. Without wishing to be bound by any particular theory, it is contemplated that the polyanion in contemplated formulations acts as a functional adhesive that anchors the remineralizing agent(s) to the tooth surface and to slowly release the bonded calcium ions from the remineralizing agents into the enamel and/or dentin. Additionally, the present disclosure provides a method for long-term remineralization, for example through dispensing the remineralization composition inside a mouth tray or a strip that is capable of holding the remineralization composition on teeth or in contact with teeth for at least 5 minutes.

In an aspect of the disclosure, a remineralization composition is provided, comprising a remineralizing agent, a polyacrylic acid sodium salt serving as a multi-point anchor to localize the remineralizing agent on tooth surface, and optionally a non-ionic surfactant. It is contemplated that, when included, the non-ionic surfactant promotes formation of complexes between the remineralizing agent and the polyacrylic acid sodium salt. In some embodiments, the composition is a ready to use gel or paste composition that does not require any steps (e.g., dissolving in water) prior to application or use. In some embodiments, the composition is formulated for remineralization of enamel and dentin. In some embodiments, the composition is free or essentially free of phosphate salts and phosphates. In some embodiments, the remineralizing agent comprises at least one of a calcium compound and a calcium complex. In some embodiments, the formation of complexes inhibits calcium ions from forming a crystalized hydroxyapatite (HA) layer on top of the tooth enamel. In some embodiments, the remineralizing agent comprises at least one of bioactive calcium containing compounds, bioactive calcium containing complexes, and free calcium ions formed once the bioactive calcium containing compounds contacts or mixes with water. In some embodiments, the remineralizing agent comprises calcium L-lactate hydrate and is present in an amount of between 2-4 wt % of the composition, and wherein the polyacrylic acid sodium salt is present in an amount of between 4-10 wt % of the composition. In some embodiments, the remineralizing agent comprises calcium formate and is present in an amount of between 1-2.5 wt % of the composition, and wherein the polyacrylic acid sodium salt is present in an amount of between 2-10 wt % of the composition. In some embodiments, the remineralizing agent comprises bioglass and is present in an amount of between 2-10 wt % of the composition, and wherein the polyacrylic acid sodium salt is present in an amount of between 2-10 wt % of the composition. In some embodiments, the composition has a pH of 6.5 to 8.5. In some embodiments, the composition is a viscous gel. In some embodiments, where present, the non-ionic surfactant is a non-ionic hydrophilic polymer surfactant comprising at least one of polyethylene glycol (PEG), polyethylene oxide (PEO), polyvinyl pyrrolidone (PVP), polyoxy-ethylene alkyl ether, a Tween surfactant, polyoxyethylene sorbitan monooleate, a derivative of ethanolamine, a di-block polyethylene oxide and polypropylene oxide (PEO-PPO) copolymer, a di-block polyethylene oxide and poly (butylene oxide) (PEO-PBO) copolymer, a tri-block PEO-PPO-PEO copolymer, a tri-block PEO-PBO-PEO copolymer, and derivatives thereof. The non-ionic surfactant, where present, may be present in any suitable amount in a remineralization composition. For example, the non-ionic surfactant(s) may comprise between 0.001 to 5 wt %, between 0.1 to 3 wt %, or between 0.2 to 2 wt % of the composition. In some embodiments, the remineralizing agent comprises at least one of a water-soluble calcium salt, a partially water-soluble calcium compound selected from calcium citrate, calcium citrate malate, calcium formate, calcium gluconate, calcium lactate, calcium nitrate, and calcium oxide, and a calcium complex selected from tricalcium oxide silicate, dicalcium oxide silicate, and a bioactive glass. In some embodiments, the remineralization composition comprises at least 15%, at least 20%, or at least 25% water by weight of the total composition weight and is free and/or essentially free of phosphates, and the remineralizing agent(s) comprise and/or consist of water-soluble calcium salt and/or at least partially water-soluble calcium compounds and/or calcium complexes, wherein the remineralizing agent(s) are present in an amount of between 2-10%, or at least 2%, 3%, 4% or at least 5% by weight of the total composition weight. One contemplated benefit of such a composition is the presence of immediate available calcium ions (e.g., formed by the calcium compounds/complexes in the presence of water), without the formation of HA that would need to be dissolved prior to use.

In another aspect of the disclosure, a remineralization composition is provided comprising a remineralizing agent and a polyanion serving as a multi-point anchor to localize the remineralizing agent to tooth surface. In some embodiments, the composition further comprises a non-ionic surfactant. In some embodiments, the remineralizing agent comprises at least one of bioactive calcium compounds and bioactive calcium complexes. In some embodiments, the at least one of bioactive calcium compounds and bioactive calcium complexes is present at a concentration of between 0.1% to 15%, between 1% to 12%, between 2% to 10%, or between 0.5% to 10% by weight of the total composition weight. In some embodiments, wherein the polyanion comprises at least one of sodium hyaluronate, sodium carboxymethyl cellulose (Na—CMC), homo- and co-polymers of alkanoic acid, homo- and co-polymers of acrylic acid, copolymers of acrylic acid and maleic acid, homo- and co-polymers of 2-acrylamido-2-methyl-1-propanesulfonic acid, homo and co-polymers of (meth)acrylated amino acid, homo- and co-polymers of vinylphosphonic acid, and derivatives and salts thereof. In some embodiments, the polyanion comprises at least one of a co-polymer of vinylphosphonic acid, a polymeric sulfonic acid, or poly(acrylic) acid, and salts thereof. In some embodiments, the polyanion is present at a concentration of between 0.1% to 15%, between 0.1% to 12%, between 0.1% to 10%, or between 0.1% to 5% by weight of the total composition weight. In some embodiments, the composition has a pH of between 6 and 12. In some embodiments, the composition has a pH of between 6.5 and 8.5. In some embodiments, the surfactant is a non-ionic hydrophilic polymer surfactant comprising at least one of polyethylene glycol (PEG), polyethylene oxide (PEO), polyvinyl pyrrolidone (PVP), polyoxy-ethylene alkyl ether, a Tween surfactant, polyoxyethylene sorbitan monooleate, a derivative of ethanolamine, a di-block polyethylene oxide and polypropylene oxide (PEO-PPO) copolymer, a di-block polyethylene oxide and poly (butylene oxide) (PEO-PBO) copolymer, a tri-block PEO-PPO-PEO copolymer, a tri-block PEO-PBO-PEO copolymer, and derivatives thereof In some embodiments, the derivative of ethanolamine comprises at least one of 2-dimethylaminoethanol and polyethoxylated tallow amine. In some embodiments, the non-ionic surfactant is present at a concentration of between 0.1% to 5% by weight of the total composition weight. In some embodiments, the composition comprises a water-miscible humectant selected from glycerin, propylene glycol, dipropylene glycol, polyethylene glycol 200, polyethylene glycol 400, polyethylene glycol 600, and combinations thereof. In some embodiments, the composition comprises desensitizing agents selected from potassium nitrate, sodium citrate, potassium citrate, sodium fluoride, calcium hydroxide, and combinations thereof. The desensitizing agent, where included, can be provided in any suitable amount in a remineralization composition. For example, the desensitizing agent may comprise between 1-20 wt %, or between 5-10 wt % of the composition. In some embodiments, the composition comprises one or more sweeteners. The sweetener can include, for example, a sugar-less agent, a sugar-free agent, an artificial sweetener, or a combination thereof, such as sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, stevia, and cyclamate salts. In some embodiments, the sweetener can be selected from stevia extract, xylitol, aspartame, sodium saccharin, and combinations thereof. In some embodiments, the composition comprises flavoring agents selected from citrus flavors, mint, berries, and combinations thereof In some embodiments, the polyanion is poly(acrylic acid) sodium salt in an amount of 2% to 10% by weight of the total composition weight, wherein the remineralizing agent is calcium L-lactate hydrate in an amount of 2% to 4% by weight of the total composition weight, and wherein the pH is 6.5 to 7.5. In some embodiments, the polyanion is poly(acrylic acid) sodium salt in an amount of 2% to 10% by weight of the total composition weight, wherein the remineralizing agent is calcium formate in an amount of 1% to 2.5% by weight of the total composition weight, and wherein the pH is 6.5 to 7.5. In some embodiments, the polyanion is poly(acrylic acid) sodium salt in an amount of 2% to 10% by weight of the total composition weight, wherein the remineralizing agent is bioglass in an amount of 2% to 10% by weight of the total composition weight, wherein the nonionic hydrophilic polymer surfactant is tri-block PEO-PPO-PEO copolymer in an amount of 0.2% to 2% by weight of the total composition weight, and wherein the pH is 6.5 to 7.5. In some embodiments, the formulation has the form of a gel, paste, cream, balm, powder, composition carried on a mouthtray or mouth-strip or adhesive patch/strip, or composition mixed with adhesive for adhesive remineralization strips. In some embodiments, the remineralizing agent comprises at least one of a water-soluble calcium salt, a partially water-soluble calcium compound selected from calcium citrate, calcium citrate malate, calcium formate, calcium gluconate, calcium lactate, calcium nitrate, and calcium oxide, and a calcium complex selected from tricalcium oxide silicate, dicalcium oxide silicate, and a bioactive glass. In some embodiments, the remineralization composition comprises at least 15%, at least 20%, or at least 25% water by weight of the total composition weight and is free and/or essentially free of phosphates, and the remineralizing agent(s) comprise and/or consist of water-soluble calcium salt and/or at least partially water-soluble calcium compounds and/or complexes, wherein the remineralizing agent(s) are present in an amount of between 2-10% by weight of the total composition weight. One contemplated benefit of such a composition is the presence of immediately available calcium ions (e.g., formed by the calcium compounds/complexes in the presence of water), without the formation of HA that would need to be dissolved prior to use.

While the disclosure herein is generally directed to compositions free of phosphates and phosphate salts, it should be appreciated that in some embodiments, a remineralization composition can comprise phosphates and/or phosphate salts, for example, where the remineralizing agent comprises amorphous calcium phosphate (ACP), amorphous calcium fluoride phosphate (ACFP), and/or tricalcium phosphate (TCP).

In another aspect of the disclosure, a method for applying a remineralization composition onto a tooth surface is provided. The remineralization composition can comprise any of the remineralization compositions described in this application, including compositions comprising a remineralizing agent, and a polyanion serving as a multi-point anchor to localize the remineralizing agent to tooth surface. Contemplated methods can comprise painting a thin layer (e.g., a layer having a thickness of between 0.5-3.0 mm, between 1.0-2.5 mm) of the remineralization composition onto a tooth surface. In some embodiments, contemplated methods can comprise following at least five minutes after painting the thin layer, washing away the remineralization composition with water. In some embodiments, contemplated methods can comprise placing at least one of a mouth tray, a mouth strip, a night guard, and an orthodontic aligner over the tooth surface after painting the thin layer. In some embodiments, the remineralization composition is formulated and applied to remain on the tooth surface, maintaining continuous remineralization for a period of at least 5 minutes while the at least one of the mouth tray, mouth strip, night guard, and orthodontic aligner is worn. In some embodiments, the remineralization composition is formulated and applied to remain on the tooth surface, maintaining continuous remineralization for a period of at least 30 minutes while the at least one of the mouth tray, mouth strip, night guard, and orthodontic aligner is worn. In some embodiments, contemplated methods can comprise wiping away the remineralization composition prior to washing away the remineralization composition.

Other advantages and benefits of the disclosed compositions, kits, and systems will be apparent to one of ordinary skill with a review of the following detailed description and accompanying drawings.

### BRIEF DESCRIPTION OF THE FIGURES

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing of which:

FIG. 1 is a flowchart of an exemplary method for applying a remineralization composition.

### DETAILED DESCRIPTION

After reading this description, it will become apparent to one skilled in the art how to implement the disclosed compositions, kits, and systems in various alternative embodiments and alternative applications. However, all the various embodiments of the present disclosure will not be described herein. It is understood that the embodiments presented here are presented by way of an example only, and not limitation. As such, this detailed description of various alternative embodiments should not be construed to limit the scope or breadth of the present disclosure as set forth below.

Unless defined otherwise, all technical and scientific terms used herein have the same meaning as commonly understood by one of ordinary skill in the art to which this disclosure belongs. Any methods and materials similar or equivalent to those described herein can be used in the practice or testing of the disclosure, as it will be understood that modifications and variations are encompassed within the spirit and scope of the instant disclosure.

As used herein, the term “a tooth structure” refers to any feature or features of a tooth including but not limited to enamel, dentin, pulp, tooth root structure, cementum, root dentin, coronal dentin, and any dental tissues or combinations thereof.

As used herein, “remineralization” of a tooth is a process whereby calcium and/or phosphate ions, and/or compounds containing calcium and/or phosphate ions, are supplied to the tooth structure from a source external to the tooth structure (underneath the outer surface of the enamel). Remineralization may be applied to restore minerals in demineralized tooth structure.

As used herein, the term “bioactive” means having a biological effect and “bioactive calcium ions” means calcium ions that are capable of bonding with hard tissue when contacted with, included in, or reacted with physiological fluids such as saliva. This bonding can naturally occur due to, for example, the concentration difference. The high concentration of remineralization agent in some contemplated compositions spontaneously converts to minerals when it reacts with phosphate in the saliva.

As used herein, the term “polyanion” or “negatively charged polymer” means a polymer with multiple negative charges.

As used herein, the terms “non-ionic surfactant”, also called “non-ionic surface active agent,” means a compound, molecule, or material that have polar head groups that are not electrically charged and can lower the surface tension between two liquids, or between a liquid and a solid. Non-ionic surfactant molecules can concentrate at the interfaces between bodies or liquid droplets.

As used herein, the term “essentially free of phosphates” means that phosphates, if any, make up no more than 0.5 wt % of the composition or component that is essentially free of phosphates, and the term “essentially free of phosphate salts” means that phosphate salts, if any, make up no more than 0.5 wt % of the composition or component that is essentially free of phosphate salts.

Tooth demineralization occurs for various reasons, including but not limited to consumption of sugary or acidic food and drinks, as well as the presence of cariogenic microbes such as Streptococcus mutans, Streptococcus sobrinus, and lactobacilli. Treatment of tooth demineralization has been attempted using highly soluble calcium and phosphate compounds capable of delivering calcium and phosphate at high concentrations. However, such compounds can be rapidly cleared by saliva. They cannot be persistently localized at the tooth surface and produce effective concentration gradient for diffusion into the subsurface of enamel. Additionally, soluble calcium and phosphate ions can only be used at low concentrations due to the intrinsic low solubility of calcium phosphate, particularly calcium fluoride phosphate. Insoluble calcium phosphate cannot be localized at the tooth surface or effectively diffuse into deeper layers of the tooth. Amorphous calcium phosphate (ACP) or amorphous calcium fluoride phosphate (ACFP) is an essential mineral phase formed in mineralized tissues and is a potential remineralizing agent in dental applications. However, the efficacy of its remineralization remains in doubt because ACP/ACFP transforms to a poorly soluble phase in saliva, and in doing so may promote dental calculus. Therefore, tooth remineralization remains a challenging and difficult task, and an unmet medical need.

In the present disclosure, polyanions are used to deliver agents that induce or provide remineralization to the teeth. In some embodiments, such delivery results in slow release of remineralizing agents at the teeth, greater persistence and/or residence time of such remineralizing agents at the teeth, and/or greater penetration of such remineralizing agents into the teeth when compared to known remineralization compositions. These effects result in effective remineralization of the teeth. In some embodiments, the polyanions bind to the remineralizing agents through electrostatic interactions or chemical bonds such as ionic bonds, which can allow for the slow and/or sustained release and long term deposition of the remineralizing agent on teeth. In some embodiments, the polyanion and remineralizing agent are in an aqueous solution.

The present disclosure relates to a remineralization composition. The composition comprises bioadhesive polyanions serving as multi-point anchors to bind or localize remineralizing agents such as bioactive calcium ions or complexes to tooth surfaces. In certain embodiments, the compositions of the disclosure comprise a polyanionic matrix capable of forming a complex with the remineralizing agents, thus resulting in slow and/or sustained release and long-term deposition of the remineralizing agent inside of tooth structure (underneath the outer surface of the enamel), preventing the remineralizing agent from pre-precipitation on tooth surface or from being quickly washed away by saliva. In the presence of polymeric non-ionic surfactants, the polyanionic matrix can attract the remineralizing agents and form complexes through electrostatic interactions or chemical bonding, which includes ionic bonds and/or hydrogen bonds. In some embodiments, this prevents calcium ions from forming a crystallized hydroxyapatite (HA) layer on the enamel surface, thus enabling the ions to migrate into the deeper layers of teeth. The method of the disclosure also relates to methods of preparing and applying said compositions for dental remineralization.

In some aspects, compositions for dental remineralization are provided including for both enamel and dentin remineralization, where the compositions comprise bioactive compounds such as calcium compounds and/or calcium complexes, polyanions and a non-ionic surfactant. In some aspects, the bioactive calcium compounds and/or complexes serve as remineralizing agents. In some aspects, the polyanions serve as multi-point anchor to adhere and or localize remineralizing agents to tooth surfaces. In some aspects, the non-ionic surfactant helps or promotes formation of complexes between the remineralizing agents and polyanions, inhibiting calcium ions from forming a crystallized hydroxyapatite (HA) layer on the enamel surface. When crystalized HA forms too quickly, it is a surface solid that can easily be removed, for example, while brushing teeth. For remineralization of teeth, calcium can be present in a water soluble form (e.g., solubilized by polyanions in liquid complex form), and the liquid can penetrate into the enamel and/or dentin.

The remineralizing agent can comprise any compound capable of binding to and being released from a polyanion to induce remineralization. In some embodiments, the remineralization agents are cations or compounds containing cations. In some embodiments, the remineralization agents are divalent cations or compounds containing divalent cations. In some embodiments, the remineralizing agents are bioactive calcium ions and/or compounds or complexes containing bioactive calcium. In some embodiments, the remineralizing agents in a composition do not include phosphates and/or phosphate salts. Such remineralizing agents include, but are not limited to, water-soluble ionic calcium or free calcium ions formed once a compound containing bioactive calcium contacts or mixes with water, or a mixture of both. In some embodiments, the compounds containing bioactive calcium are water-soluble calcium salts; partially water-soluble compounds including but not limited to calcium citrate, calcium citrate malate, calcium formate, calcium gluconate, calcium lactate, and/or calcium nitrate; calcium complex compounds including but limited to tricalcium oxide silicate and/or dicalcium oxide silicate; and/or bioactive glass comprising SiO₂, CaO, Na₂O, P₂O₅, CaF₂, B₂O₃, K₂O, and/or MgO. Any mixture of these can be used. In some embodiments, mixtures of water-soluble calcium salts and calcium-containing complexes are used. In some embodiments, the poly(acrylic acid) (PAA) stabilizes the calcium salts. In some embodiments, the concentration of bioactive calcium in the composition, whether present as free calcium ions or in a compound, can be at least 0.05%, at least 1%, at least 2%, at least 3%, at least 4%, at least 5%, 0.001% to 30%, 0.05% to 20%, 0.01% to 10%, or 0.05% to 5% by weight of the total composition weight to ensure effective remineralization. In a preferred embodiment, the bioactive calcium content in the remineralization composition is 0.05% to 5%.

In some embodiments, the bioactive calcium is capable of forming or participating in forming calcium-containing complexes that restore mineral ions when placed in the aqueous environment of the tooth, thereby remineralizing the tooth. Calcium and phosphate ions then migrate into deep layers of the tooth, forming a calcium-phosphate rich surface layer. Over time, the calcium-phosphate rich layer crystallizes into hydroxyapatite. Collagen can become structurally integrated with the apatite agglomerates. An effective remineralizing amount is any amount capable of forming hydroxyapatite within tooth structure (underneath an outer surface of enamel).

The polyanion of the present disclosure can be any polyanion capable of binding to and releasing a remineralization agent. In some embodiments, the polyanion is soluble or hydrated in water (hydrophilic) and has an appropriate molecular weight and a sufficiently high charge density to attract cationic remineralization agents such as calcium ions, calcium compounds, or calcium complexes to form complexes. Such complexes are formed through either electrostatic interactions or chemical bonding, including ionic bonds and hydrogen bonds or both. This prevents the calcium ions in the remineralization composition and phosphate ions existing in saliva from forming a crystallized hydroxyapatite (HA) layer on the surface of enamel, thereby enabling the calcium ions/complexes to migrate into the deeper layers of tooth.

In some aspects, the polyanion of the compositions of the present disclosure is a negatively charged polymer, wherein the polymer can be natural or synthetic, or a mix of natural and synthetic polymers. A natural polymer can be a polymer found in nature but synthetically manufactured. A synthetic polymer can be a polymer not found in nature. In some embodiments, the natural polymer is alginate, or sodium hyaluronate. In other embodiments, the polyanions are synthetic polymers. In particular, contemplated polyanions include, but are not limited to, sodium carboxymethyl cellulose (Na—CMC); homo- or co-polymer of alkanoic acid; homo- or copolymer of acrylic acid; copolymer of acrylic acid and maleic acid; homo- or co-polymer of 2-acrylamido-2-methyl-1-propanesulfonic acid; homo or co-polymer of (meth)acrylated amino acid; homo- or co-polymer of vinylphosphonic acid. In some embodiments, the negatively charged polymer is a polycarboxylate, a methyl vinyl ether/maleic anhydride copolymer and the like, alone and mixtures thereof. The polycarboxylate may also be cross-linked, e.g., with polyalkenyl ethers or divinyl glycol. The polymers may be used alone or in combinations thereof. Derivatives and/or salts, including but not limited to ammonium or sodium salts of the polymers, may also be used. While the examples herein are generally directed to compositions having poly(acrylic acid) sodium salt as the polyanion, it is contemplated that other polyanions (e.g., the aforementioned polyanions) can be used. Poly(acrylic acid) sodium salt and some other contemplated polyanions are highly negatively charged and can bond to calcium in enamel and dentin (bioadhesive to tooth structure) as well as calcium in the composition (polyanionic complex).

Representative structures of negatively charged polymers binding to calcium ions or calcium complexes are shown in Examples 1-1, 1-2, 2-1, and 2-2. In some embodiments, the polymer is a co-polymer of vinylphosphonic acid (VPA) as shown in Examples 1-1 and 1-2, showing the schematic structure of the complex between a co-polymer of vinylphosphonic acid and calcium ions or calcium complexes, where R1 is selected from alkyl and aryl groups, m is the number of repeat units of vinyl phosphonic acid (VPA), and n is the number of repeat units of acrylate. Ca represents calcium ions and CaP represents calcium complexes. When the content of VPA increased in the co-polymer, the pH of the solution decreases and therefore, copolymers with higher VPA contents will show increased dissociation, and thus a greater negative charge density and a greater capacity to chelate (complex) calcium. In an embodiment, the negatively charged polymer forms a complex with calcium ions or adheres onto the surface of calcium complexes, such as bioglass or hydroxyapatite. In some embodiments, the polymer is polymeric sulfonic acid as shown in Examples 2-1 and 2-2 showing the schematic structure of the complex between polymeric sulfonic acid and calcium ions or calcium complexes, where n is the number of repeat units of 2-acryamido-2-methyl-1-propanesulfonic acid. Ca represents calcium ions and CaP represents calcium complexes.

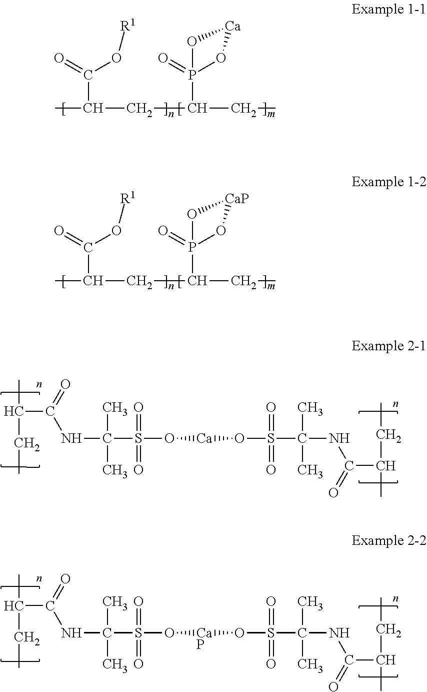

In some embodiments, the amount of polyanion is sufficient to attract bioactive calcium ions, calcium-containing compounds, and/or complexes and form nano-sized complexes. In some embodiments, the composition comprises 0.001% to 40%, 0.005% to 30%, 0.01% to 20% or 0.05% to 10% of negatively charged hydrophilic polymers by weight of the total composition weight. In a preferred embodiment, the composition comprises 0.05% to 10% of negatively charged hydrophilic polymers by weight of the total composition weight. In some embodiments, the composition contains sufficient negatively charged hydrophilic polymers to ensure that bioactive calcium ions, calcium-containing compounds, and/or complexes are adhered/chelated effectively within the remineralization composition.

The non-ionic surfactant of the present disclosure can be any non-ionic surfactant capable, in aqueous medium, of promoting formation of nano-sized complexes between the remineralization agent and the polyanion, maintaining the complexes in nano-size, and/or inhibiting crystallization or aggregation of crystals of calcium and phosphate ions and/or crystalized hydroxyapatite (HA) on the outer surface of the enamel. These effects enable the remineralization agent, such as bioactive calcium ions and calcium complexes, to migrate into the deeper layers of the tooth throughout the application period of the remineralization composition. In some embodiments, nonionic surfactants have covalently bonded oxygen-containing hydrophilic groups, which are bonded to hydrophobic parent structures. The water solubility of the oxygen groups is the result of hydrogen bonding. In some embodiments, the non-ionic surfactants contemplated include but are not limited to polyvinylpyrrolidone (PVP), alkylethers of poly(ethylene glycol) such as polyethylene glycol (PEG) or polyethylene oxide (PEO), alkylethers of poly(propylene glycol), block copolymers of alkylether of poly(ethylene glycol) such as di-block polyethylene oxide and polypropylene oxide (PEO-PPO) copolymer or tri-block PEO-PPO-PEO copolymer, di-block polyethylene oxide and poly(butylene oxide) (PEO-PBO) copolymer, tri-block PEO-PBO-PEO copolymer and the like, sugar-based nonionic surfactants such as Tween surfactants including but not limited to polyoxyethylene sorbitan monooleate, or derivatives of ethanolamine such as 2-dimethylaminoethanol and polyethoxylated tallow amine and the like. In some embodiments, the nonionic surfactants contemplated include Pluronics/poloxamers, polyethylene oxide condensates of alkyl phenols, alkylphenolethoxylates, products derived from the condensation of ethylene oxide with the reaction product of propylene oxide and ethylene diamine, ethylene oxide condensates of aliphatic alcohols, tertiary amine oxides including but not limited to long chain tertiary amine oxides, long chain tertiary phosphine oxides, long chain dialkyl sulfoxides, polyoxyethylenesorbitan esters, fatty alcohol ethoxylates, tertiary phosphine oxides, dialkylsulfoxides, and mixtures of such materials. Combinations of surfactants, including but not limited to combinations of the nonionic surfactants listed herein, may be used. In some embodiments, the amount of non-ionic surfactants is sufficient to promote chelating or complexing calcium ions in calcium-containing compounds, and/or calcium complexes with the polyanions and form nano-sized calcium complexes. In some embodiments, the composition comprises 0.001% to 10%, more preferably, 0.1% to 2% of the non-ionic surfactant(s) by weight of the total composition weight.

In some embodiments, the remineralization composition has nearly neutral to slightly basic pH, ranging from 6 to 12, from 6.5-10, from 6-9, or 6.5 to 8.5. In a preferred embodiment, the pH of the remineralization composition is 6.5 to 8.5. The pH of the composition may be adjusted according to any manner known to those skilled in the art. In some embodiments, the pH of the composition is adjusted with sodium hydroxide, potassium hydroxide, ammonium hydroxide, trimethylamine, calcium hydroxide, calcium oxide, di-calcium oxide silicate, tri-calcium oxide silicate, or mixtures thereof. However, all suitable pH adjusters are contemplated herein.

In some embodiments, the negatively charged polymers, in aqueous medium, at near-neutral pH especially at pH 6.5-8.5, serve as multi-point anchors to bond and/or adhere bioactive calcium ions through either electrostatic attraction or chemical bonding, including ionic bonding and hydrogen bonding or both, preventing the bioactive calcium ions from forming a crystallized hydroxyapatite (HA) layer on the enamel surface, and thus enabling calcium ions and/or calcium complexes to migrate into the deeper layers of the tooth.

In some embodiments, one or more optional desensitizing agents may be provided in a remineralizing composition, for example, potassium nitrate, sodium citrate, potassium citrate, sodium fluoride, calcium hydroxide, mixtures thereof, or any other suitable desensitizing agent(s).

The remineralizing compositions of the present disclosure may also include one or more optional flavoring agents or flavorings. Suitable flavorings may exhibit fragrant properties. The flavorings may be any flavors known by those skilled in the art to be used in food products or dental products. The flavorings may be natural oils, synthetic flavor oils, flavoring aromatics, extracts derived from plants, leaves, flowers, or fruits, or combinations thereof. Flavor oils which may be used include but are not limited to any one or combination of spearmint oil, cinnamon oil, peppermint oil, clove oil, bay oil, thyme oil, cedar leaf oil, oil of nutmeg, oil of sage, and oil of bitter almonds. Commonly used flavoring agents that can be used include mints such as peppermint, artificial vanilla, cinnamon derivatives, and various fruit flavors, whether employed individually or in a mixture. The flavoring agents, where present, may be present in any suitable amount in a remineralization composition. For example, the flavoring agent(s) may comprise between 0.001 to 3wt %, between 0.01 to 2 wt %, or between 0.1 to 1 wt % of the composition

The remineralizing compositions of the present disclosure may include a sweetening agent or a combination of sweetening agents. The sweetening agents may be present in any suitable amount. In some embodiments, the sweetening agent(s) may be present in an amount of less than about 5 wt %, or less than about 2 wt %. In a preferred embodiment, the sweetening agent(s) are present in an amount of less than about 2 wt %.

Suitable sweetening agents that can be included in contemplated remineralizing compositions include, for example, monosaccharides, disaccharides, and polysaccharides alone or in combination. Such monosaccharides, disaccharides, and polysaccharides include but are not limited to xylose, ribose, glucose (dextrose), mannose, galactose, fructose, (i.e., levulose), sucrose (i.e., sugar), maltose, an invert sugar (i.e., a mixture of fructose and glucose derived from sucrose), partially hydrolyzed starch, corn syrup solids, dihydrochalcones, monellin, steviosides, and glycyrrhizin. Additionally or alternatively, natural sweeteners such as stevia leaf extract and water-soluble artificial sweeteners such as soluble saccharin salts and aspartame may be used, alone or combination thereof.

The remineralization compositions of the present disclosure may optionally include one or more excipients. Excipients for use in oral hygiene and dentrifice compositions are well known in the art. Classes of excipients may include polishing material (dental abrasives), desensitizing agents, waxes, emollients, thickening agents/viscosity increasing agents (e.g., Xanthan gum, hydroxyethyl cellulose, a combination thereof), humectants, pH modifiers, water repelling agents, anti-foaming agents, surfactants, solubilizers, wetting agents, binders, fillers, antimicrobial agents, antioxidants, preservatives, buffers and buffering agents, and solvents. The excipients may be present in the composition at any suitable concentration. Suitable humectants for use in compositions of the disclosure include but are not limited to glycerin, sorbitol, xylitol, propylene glycol or polyethylene glycol, or mixtures thereof. The humectant(s) may be provided in a remineralization composition in any suitable amount, including for example, about 5-80 wt%, about 10-70 wt %, or about 20-50 wt % of the composition. Suitable preservatives for use in the compositions of the disclosure include but are not limited to parabens (methyl and propyl parabens), sodium benzoate, and potassium sorbate. The preservatives, where present, may be present in any suitable amount in a remineralization composition. For example, the preservative(s) may comprise between 0.001 to 2wt %, or between 0.1 to 1 wt % of the composition. Suitable desensitizing agents include but are not limited to physiologically or orally acceptable potassium salts, e.g., potassium nitrate or potassium chloride.

Water (e.g., deionized water) may be provided in a remineralization composition in any suitable amount, including for example, about 5-80 wt %, about 10-50 wt %, or about 20-40 wt % of the composition. In some embodiments, water is present in an amount of at least 10 wt % of the composition. In some embodiments, water is present in an amount of at least 20 wt % of the composition. In some embodiments, water is present in an amount of at least 30 wt % of the composition.

In some contemplated remineralization formulations, enzymes and the like may be provided. In some embodiments, the remineralization composition is in the form of a paste or cream, balm, powder, or non-abrasive paste or gel, a composition carried on a mouth-tray or a mouth-strip or adhesive patch/strip, and/or a composition mixed with adhesives for adhesive remineralization strips. In a preferred embodiment, the remineralization composition is in the form of a non-abrasive paste or gel.

The following examples further describe some embodiments within the scope of the present disclosure. These examples are given solely for the purpose of illustration, and are not to be construed as limitations of the present disclosure since many variations are possible without departing from its spirit and scope.

Remineralization Composition 1 describes a remineralization composition containing water-soluble calcium salt.

### Remineralization Composition 1

Composition ranges of the ingredients
in the remineralization composition

| Ingredient | % by weight |
|---|---|
| Glycerin | 20-50 |
| DI water | 20-40 |
| Potassium nitrate | 5-10 |
| Poly(acrylic acid) sodium salt | 2-10 |
| Calcium L-lactate hydrate | 2-4 |
| Potassium hydroxide solution (30% w/w) or | 2-15 |
| Trolamine | |
| Sodium saccharin | 0.1-0.4 |
| Vanilla | 0.05-0.2 |
| Methylparaben | 0.025 |
| Propylparaben | 0.01 |

Remineralization Composition 1 was a viscous gel. The pH was measured using a pH meter. The pH was 6.5 to 7.5. The calcium concentration was determined by colorimetric titration with disodium ethylene diaminetetraacetate (EDTA) volumetric solution. The calcium concentration in the composition was 0.2-0.6%. Viscosity was measured with a Brookfield Rheometer. The viscosity of the example composition was 40,000 centipoises to 120,000 centipoises (cps).

Remineralization Composition 2 describes a remineralization composition containing another water-soluble calcium salt.

### Remineralization Composition 2

Composition ranges of the ingredients
in remineralization composition

| Ingredients | % by weight |
|---|---|
| Glycerin | 20-50 |
| DI water | 20-40 |
| Potassium nitrate | 5-10 |
| Poly(acrylic acid) sodium salt | 2-10 |
| Calcium formate | 1-2.5 |
| Potassium hydroxide solution (30% w/w) or | 2-15 |
| Trolamine | |
| Sodium saccharin | 0.1-0.4 |
| Vanilla | 0.05-0.2 |
| Methylparaben | 0.025 |
| Propylparaben | 0.01 |

Remineralization Composition 2 was a clear viscous gel. The pH was measured with a pH meter. The pH was 6.5 to 7.5. The calcium concentration was determined by colorimetric titration with disodium ethylene diaminetetraacetate (EDTA) volumetric solution. The calcium concentration in the composition was 0.3%-0.7%. Viscosity was measured with a Brookfield Rheometer. The viscosity of the example composition was 40,000 centipoises to 120,000 centipoises (cps).

Remineralization Composition 3 describes a remineralization composition containing bioactive glass.

### Remineralization Composition 3

Composition ranges of the ingredients
in remineralization composition

| Ingredients | % by weight |
|---|---|
| Glycerin | 20-50 |
| DI water | 20-40 |
| Potassium nitrate | 5-10 |
| Poly(acrylic acid) sodium salt | 2-10 |
| Bioglass | 2-10 |
| Potassium hydroxide solution (30% w/w)/ | 2-15 |
| Trolamine | |
| Tri-block PEO-PPO-PEO copolymer | 0.2-2 |
| Sodium saccharin | 0.1-0.4 |
| Vanilla | 0.05-0.5 |
| Methylparaben | 0.025 |
| Propylparaben | 0.01 |

Remineralization Composition 3 was an opaque viscous paste. The pH was measured with a pH meter. The pH was 6.5 to 7.5. The calcium concentration was determined by colorimetric titration with disodium ethylene diaminetetraacetate (EDTA) volumetric solution. The calcium concentration in the composition was 0.02%-0.2%. Viscosity was measured with a Brookfield Rheometer. The viscosity of the example composition was 40,000 centipoises to 120,000 centipoises (cps).

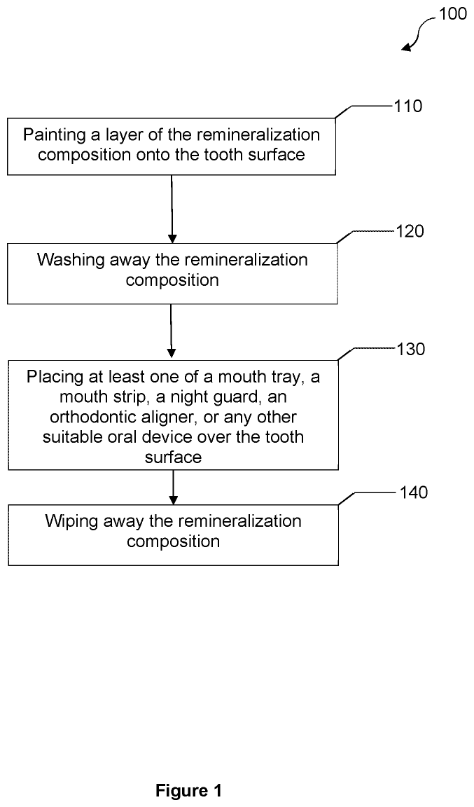

The remineralization compositions described herein, including but not limited to, Remineralization Compositions 1-3 above, can be applied, used and/or removed using any suitable method(s). FIG. 1 illustrates an exemplary method 100 of the disclosure. A step of method 100 can comprise painting a layer (e.g., a thin layer, for example, 0.5-3.0 mm layer) of the remineralization composition onto the tooth surface, as shown in Step 110. The remineralization composition can comprise a remineralizing agent, and a polyanion serving as a multi-point anchor to localize the remineralizing agent. Another step of method 100 can comprise washing away the remineralization composition, as shown in Step 120. The remineralization composition can be washed away with water or other suitable rinse. In some embodiments, washing away the remineralization composition occurs at least five minutes after painting the layer of the remineralization composition onto the tooth surface. Another step of method 100 can comprise placing at least one of a mouth tray, a mouth strip, a night guard, an orthodontic aligner, or any other suitable oral device over the tooth surface, as shown in Step 130. In some embodiments, Step 130 can take place after Step 110 and before Step 120. In some embodiments, the remineralization composition is formulated and applied to remain on the tooth surface, maintaining continuous remineralization for a period of at least 5 minutes, at least 10 minutes, at least 15 minutes, at least 20 minutes, at least 25 minutes, or at least 30 minutes while the oral device is worn. Another step of method 100 can comprise wiping away the remineralization composition, as shown in Step 140. Step 140 can occur, for example, prior to washing away the remineralization composition.

In another embodiment, a method of using a remineralization composition comprises: placing a suitable amount of the remineralization composition, formulated as a toothpaste, onto a toothbrush; brushing teeth for at least two minutes; and rinsing with water to wash away the remineralization composition from the teeth.

In an embodiment, a method of applying the tooth remineralization composition enables the remineralization composition to remain on the tooth surface, maintaining continuous remineralization while a mouth tray/strip, night guard or orthodontic aligner is worn.

### Non-Limiting Embodiments.

Embodiment 1. A remineralization composition, comprising a remineralizing agent, and a poly(acrylic acid) sodium salt serving as a multi-point anchor to localize the remineralizing agent(s) to tooth surfaces, wherein the remineralizing composition is formulated as a gel or paste, and free of phosphates.

Embodiment 2. The remineralization composition of embodiment 1, further comprising a non-ionic surfactant, wherein the non-ionic surfactant promotes formation of complexes between the remineralizing agent(s) and the poly(acrylic acid) sodium salt.

Embodiment 3. The remineralization composition of any of embodiments 1-2, wherein the composition is formulated for remineralization of enamel and dentin.

Embodiment 4. The remineralization composition of any of embodiments 1-3, wherein the composition is formulated as a ready to use composition.

Embodiment 5. The remineralization composition of any of embodiments 1-4, wherein the remineralization agent comprises at least one of a calcium compound and a calcium complex.

Embodiment 6. The remineralization composition of any of embodiments 1-5, wherein the formation of complexes inhibits calcium ions from forming a pre-crystalized hydroxyapatite (HA) layer at a surface of an enamel of a tooth.

Embodiment 7. The remineralization composition of any of embodiments 1-6, wherein the remineralizing agent comprises at least one of bioactive calcium containing compounds, bioactive calcium containing complexes, and free calcium ions formed once the bioactive calcium containing compounds contacts or mixes with water.

Embodiment 8. The remineralization composition of any of embodiments 1-7, wherein the remineralizing agent comprises calcium L-lactate hydrate and is present in an amount of between 2-4 wt % of the composition, and wherein the poly(acrylic acid) sodium salt is present in an amount of between 4-10 wt % of the composition.

Embodiment 9. The remineralization composition of any of embodiments 1-8, wherein the remineralizing agent comprises calcium formate and is present in an amount of between 1-2.5 wt % of the composition, and wherein the poly(acrylic acid) sodium salt is present in an amount of between 2-10 wt % of the composition.

Embodiment 10. The remineralization composition of any of embodiments 1-9, wherein the remineralizing agent comprises bioglass and is present in an amount of between 2-10 wt % of the composition, and wherein the poly(acrylic acid) sodium salt is present in an amount of between 2-10 wt % of the composition.

Embodiment 11. The remineralization composition of any of embodiments 1-10, wherein the composition has a pH of 6.5 to 8.5.

Embodiment 12. The remineralization composition of any of embodiments 1-11, wherein the composition is a viscous gel.

Embodiment 13. The remineralization composition of any of embodiments 1-12, wherein the surfactant is a non-ionic hydrophilic polymer surfactant comprising at least one of polyethylene glycol (PEG), polyethylene oxide (PEO), polyvinyl pyrrolidone (PVP), polyoxy-ethylene alkyl ether, a Tween surfactant, polyoxyethylene sorbitan monooleate, a derivative of ethanolamine, a di-block polyethylene oxide and polypropylene oxide (PEO-PPO) copolymer, a di-block polyethylene oxide and poly (butylene oxide) (PEO-PBO) copolymer, a tri-block PEO-PPO-PEO copolymer, a tri-block PEO-PBO-PEO copolymer, and derivatives thereof.

Embodiment 14. The remineralization composition of any of embodiments 1-13, wherein the remineralizing agent comprises at least one of a water-soluble calcium salt, a partially water-soluble calcium compound selected from calcium citrate, calcium citrate malate, calcium formate, calcium gluconate, calcium lactate, calcium nitrate, and calcium oxide, and a calcium complex selected from tricalcium oxide silicate, dicalcium oxide silicate, and a bioactive glass.

Embodiment 15. A remineralization composition, comprising a remineralizing agent, and a polyanion serving as a multi-point anchor to localize the remineralizing agent.

Embodiment 16. The remineralization composition of embodiment 15, further comprising a non-ionic surfactant.

Embodiment 17. The remineralization composition of any of embodiments 15-16, wherein the remineralizing agent comprises at least one of bioactive calcium compounds and bioactive calcium complexes.

Embodiment 18. The remineralization composition of any of embodiments 15-17, wherein the at least one of bioactive calcium compounds and bioactive calcium complexes is present at a concentration of 0.5% to 10% by weight of the total composition weight.

Embodiment 19. The remineralization composition of any of embodiments 15-18, wherein the polyanion comprises at least one of sodium hyaluronate, sodium carboxymethyl cellulose (Na-CMC), homo- and co-polymers of alkanoic acid, homo- and co-polymers of acrylic acid, copolymers of acrylic acid and maleic acid, homo- and co-polymers of 2-acrylamido-2-methyl-1-propanesulfonic acid, homo and co-polymers of (meth)acrylated amino acid, homo- and co-polymers of vinylphosphonic acid, and derivatives and salts thereof.

Embodiment 20. The remineralization composition of any of embodiments 15-19, wherein the polyanion comprises at least one of a co-polymer of vinylphosphonic acid, a polymeric sulfonic acid, or poly(acrylic) acid, and salts thereof

Embodiment 21. The remineralization composition of any of embodiments 15-20, wherein the polyanion is present at a concentration of between 0.1% to 10% by weight of the total composition weight.

Embodiment 22. The remineralization composition of any of embodiments 15-21, wherein the composition has a pH of 6 to 12 and is free or essentially free of phosphates.

Embodiment 23. The remineralization composition of any of embodiments 15-22, wherein the composition has a pH of 6.5 to 8.5.

Embodiment 24. The remineralization composition of any of embodiments 15-23, wherein the surfactant is a non-ionic hydrophilic polymer surfactant comprising at least one of polyethylene glycol (PEG), polyethylene oxide (PEO), polyvinyl pyrrolidone (PVP), polyoxy-ethylene alkyl ether, a Tween surfactant, polyoxyethylene sorbitan monooleate, a derivative of ethanolamine, a di-block polyethylene oxide and polypropylene oxide (PEO-PPO) copolymer, a di-block polyethylene oxide and poly (butylene oxide) (PEO-PBO) copolymer, a tri-block PEO-PPO-PEO copolymer, a tri-block PEO-PBO-PEO copolymer, and derivatives thereof

Embodiment 25. The remineralization composition of any of embodiments 15-24, wherein the derivative of ethanolamine comprises at least one of 2-dimethylaminoethanol and polyethoxylated tallow amine.

Embodiment 26. The remineralization composition of any of embodiments 15-25, wherein the surfactant is present at a concentration of between 0.1% to 5% by weight of the total composition weight.

Embodiment 27. The remineralization composition of any of embodiments 1-26, wherein the remineralization composition has a viscosity of at least 45,000 centipoises.

Embodiment 28. The remineralization composition of any of embodiments 1-26, wherein the remineralization composition has a viscosity of at least 50,000 centipoises.

Embodiment 29. The remineralization composition of any of embodiments 1-28, wherein the remineralization composition comprises at least 20% water by weight of the total composition weight.

Embodiment 30. The remineralization composition of any of embodiments 1-28, wherein the remineralization composition comprises at least 25% water by weight of the total composition weight.

Embodiment 31. A method of applying a remineralization composition of any of embodiments 1-30, comprising painting a thin layer of the remineralization composition to a tooth surface, and following at least five minutes after painting the thin layer, washing away the remineralization composition with water.

Embodiment 32. A method of applying a remineralization composition to a tooth surface, comprising: painting a thin layer of the remineralization composition onto a tooth surface, the remineralization composition comprising: a remineralizing agent; and a polyanion serving as a multi-point anchor to localize the remineralizing agent; and following at least five minutes after painting the thin layer, washing away the remineralization composition with water.

Embodiment 33. The method of any of embodiments 31-32, further comprising placing at least one of a mouth tray, a mouth strip, a night guard, and an orthodontic aligner over the tooth surface after painting the thin layer.

Embodiment 34. The method of any of embodiments 31-33, wherein the remineralization composition is formulated and applied to remain on the tooth surface, maintaining continuous remineralization for a period of at least 5 minutes while the at least one of the mouth tray, mouth strip, night guard, and orthodontic aligner is worn.

Embodiment 35. The method of any of embodiments 31-34, wherein the remineralization composition is formulated and applied to remain on the tooth surface, maintaining continuous remineralization for a period of at least 30 minutes while the at least one of the mouth tray, mouth strip, night guard, and orthodontic aligner is worn.

Embodiment 36. The method of any of embodiments 31-35, further comprising wiping away the remineralization composition prior to washing away the remineralization composition.

Thus, specific examples of remineralization compositions, kits and methods have been disclosed. It should be apparent, however, to those skilled in the art that many more modifications besides those already described are possible without departing from the inventive concepts herein. While examples and variations of the many aspects of the invention have been disclosed and described herein, such disclosure is provided for purposes of explanation and illustration only. Thus, various changes and modifications may be made without departing from the scope of the claims.

It will be understood that the benefits and advantages described above may relate to one embodiment or may relate to several embodiments. Aspects described in connection with one embodiment are intended to be able to be used with the other embodiments. Any explanation in connection with one embodiment applies to similar features of the other embodiments, and elements of multiple embodiments can be combined to form other embodiments. The embodiments are not limited to those that solve any or all of the stated problems or those that have any or all of the stated benefits and advantages.

As used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Reference throughout this specification to “an embodiment” or “an implementation” means that a particular feature, structure, or characteristic described in connection with the embodiment is included in at least one embodiment or implementation. Thus, appearances of the phrases “in an embodiment” in various places throughout this specification are not necessarily all referring to the same embodiment or a single exclusive embodiment. Furthermore, the particular features, structures, or characteristics described herein may be combined in any suitable manner in one or more embodiments or one or more implementations.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Certain numerical values and ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, and/or C. For example, a combination of A and B may comprise one A and multiple B's, multiple A's and one B, or multiple A's and multiple B's.

All structural and functional equivalents to the components of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A remineralization composition, comprising:
a remineralizing agent;
a poly(acrylic acid) sodium salt serving as a multi-point anchor to localize the remineralizing agent on tooth surfaces; and
wherein the remineralizing composition is formulated as a gel or paste, and is free of phosphates.

2. The composition of claim 1, further comprising a non-ionic surfactant, wherein the non-ionic surfactant promotes formation of complexes between the remineralizing agent and the poly(acrylic acid) sodium salt.

3. The composition of claim 1, wherein the composition is formulated for remineralization of enamel and dentin.

4. The composition of claim 1, wherein the composition is formulated as a ready to use composition.

5. The composition of claim 1, wherein the remineralization agent comprises at least one of a calcium compound and a calcium complex.

6. The composition of claim 1, wherein the formation of complexes inhibits calcium ions from forming a crystalized hydroxyapatite (HA) layer at an enamel surface of a tooth.

7. The composition of claim 1, wherein the remineralization composition comprises at least 20% water by weight of the total composition weight, and wherein the remineralizing agent comprises at least one of bioactive calcium containing compounds, bioactive calcium containing complexes, and free calcium ions formed once the bioactive calcium containing compounds contacts or mixes with the water.

8. The composition of claim 1, wherein the remineralizing agent comprises calcium L-lactate hydrate and is present in an amount of between 2-4wt % of the composition, and wherein the poly(acrylic acid) sodium salt is present in an amount of between 4-10 wt % of the composition.

9. The composition of claim 1, wherein the remineralizing agent comprises calcium formate and is present in an amount of between 1-2.5 wt % of the composition, and wherein the poly(acrylic acid) sodium salt is present in an amount of between 2-10 wt % of the composition.

10. The composition of claim 1, wherein the remineralizing agent comprises bioglass and is present in an amount of between 2-10 wt % of the composition, and wherein the poly(acrylic acid) sodium salt is present in an amount of between 2-10 wt % of the composition.

11. The composition of claim 1, wherein the composition has a pH of 6.5 to 8.5.

12. The composition of claim 1, wherein the composition is a viscous gel.

13. The composition of claim 8, wherein the composition has a pH of 6.5 to 8.5, and is a viscous gel.

14. The composition of claim 9, wherein the composition has a pH of 6.5 to 8.5, and is a viscous gel.

15. The composition of claim 10, wherein the composition has a pH of 6.5 to 8.5, and is a viscous gel.

16. The composition of claim 2, wherein the non-ionic surfactant is a non-ionic hydrophilic polymer surfactant comprising at least one of polyethylene glycol (PEG), polyethylene oxide (PEO), polyvinyl pyrrolidone (PVP), polyoxy-ethylene alkyl ether, a Tween surfactant, polyoxyethylene sorbitan monooleate, a derivative of ethanolamine, a di-block polyethylene oxide and polypropylene oxide (PEO-PPO) copolymer, a di-block polyethylene oxide and poly (butylene oxide) (PEO-PBO) copolymer, a tri-block PEO-PPO-PEO copolymer, a tri-block PEO-PBO-PEO copolymer, and derivatives thereof.

17. The composition of claim 1, wherein the remineralizing agent comprises at least one of a water-soluble calcium salt, a partially water-soluble calcium compound selected from calcium citrate, calcium citrate malate, calcium formate, calcium gluconate, calcium lactate, calcium nitrate, and calcium oxide, and a calcium complex selected from tricalcium oxide silicate, dicalcium oxide silicate, and a bioactive glass.

18. A remineralization composition, comprising:
a remineralizing agent; and
a polyanion serving as a multi-point anchor to localize the remineralizing agent to tooth surfaces, wherein the remineralization composition is free of phosphates.

19. The remineralization composition of claim 18, wherein the remineralization composition comprises at least 20% water by weight of the total composition weight.
